(19) United States
(12) Patent Application Publication
(10) Pub. No.: US 2016/0175377 A1
(43) Pub. Date: Jun. 23, 2016

(54) FORMULATION AND FUNCTIONALITY OF PHENOLIPIDS FOR NOVEL FOODS AND PHARMACEUTICALS

(71) Applicant: Umm Al-Qura University, Makkah (SA)

(72) Inventor: Mohamed Fawzy Ramadan HASSANIEN, Makkah (SA)

(73) Assignee: Umm Al-Qura University, Makkah (SA)

(21) Appl. No.: 14/412,350

(22) PCT Filed: Dec. 19, 2014
(86) PCT No.: PCT/IB2014/002849

§ 371 Date: Dec. 31, 2014

(51) Int. Cl. A61K 36/906; A61K 36/61; A61K 31/352; A61K 36/45; A61K 36/185; A61K 36/53; A61K 36/23

(52) U.S. Cl. CPC A61K 36/906; A61K 36/53; A61K 36/61; A61K 36/23; A61K 36/45; A61K 36/185; A61K 31/352; A23L 1/3002; A23L 1/3006; A23V 2002/00

(57) ABSTRACT

Phenolipids is a composition containing a phenolics complexed to a glycolipid or phospolipid, where phenolics could be one or more phenolic compound or a plant extract rich in phenolic compounds. A method for making a complex (Phenolipids) of a phenolics and a glycolipid or phospholipid by combining a plant extract and a glycolipid or phospholipid in a solvent medium selected from water and ethyl acetate.

### BACKGROUND OF THE DISCLOSURE

1. Technical Field

The present disclosure relates to a composition (Phenolipids) comprising a phenolics complexed to glycolipids, where phenolics are one or more phenolic compound or a plant extract rich in phenolic compounds and a method for making a complex (Phenolipids) of a phenolics and glycolipids by combining the plant extract and the glycolipid in a solvent medium selected from water and ethyl acetate.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Phenolic compounds and extracts rich in bioactive compounds from medicinal plants are studied for positive effects on health and the stability of food. In oil-containing foods, the use of phenolic comopunds or extracts can be difficult because their solubility is limited due to their high hydrophilicity. Therefore, new ways to improve the solubility of phenolic compounds or plant extracts in oil-containing food may help overcome these problems and improve the stability and the shelf-life of foods. An added value could be improved bioavailability of phenolic compounds in human nutrition resulting in a better nutritional value of food.

Bombard et al. (U.S. Pat. No. 5,043,323 A) prepared complex compounds of flavonoids with phospholipids using toxic aprotic solvents not including ethyl acetate.

Gary et al. (U.S. 2011/0280950 A1) prepared an antioxidant emulsion that includes quercetin, lecithin, and a lipid carrier. The effectiveness of the emulsion including quercetin and lecithin at stabilizing soybean oil against oxidation was compared to other commercially available additives. The results demonstrated that quercetin and lecithin antioxidant composition exerted a strong stabilizing effect upon soybean oil in a dose-dependent manner.

Another study (http://www.dissertationtopic.net/doc/1260196) prepared ginkgo phospholipids and quercetin complex using organic solvent rather than ethyl acetate.

Sun, Xu, Yang, Li, Liu, Pan and Yuan (Formulation of a stable and high-loaded quercetin injectable emulsion. Pharmaceutical Development and Technology 16(6): 609-615 —incorporated by reference in its entirety) prepared a stable and high-loaded quercetin emulsion with a quercetin-phospholipid complex. Quercetin and soybean lecithin were reacted in dichloromethane at 40° C. to prepare the complex.

Han et al. (CN 102085238 B) prepared glycosides containing phospholipid complex. The complex obtained by the invention improved the glycosides' phospholipid compound penetrating ability and biological availability of the tested biological film.

Date et al. (Lecithin-based novel cationic nanocarriers (Leciplex) II: improving therapeutic efficacy of quercetin on oral administration. Molecular Pharmcology 6: 716-726 -incorporated by reference in its entierty) evaluated the ability of novel self-assembled phospholipid-based cationic nanocarriers (LeciPlex) in improving the therapeutic efficacy of a poorly water-soluble natural polyphenolic agent-quercetin-on oral administration. Quercetin loaded LeciPlex (QR-LeciPlex) were successfully fabricated using a biocompatible solvent Transcutol HP.

Sampalis (WO 20030311873 A2) extracted phospholipid from a marine or aquatic biomass possesses therapeutic properties. The phospholipid extract comprised a variety of phospholipids, fatty acid, metals and a novel flavonoids.

Dr. Mohamed Fawzy Ramadan developed a simple procedure to prepare complexes of phenolic compounds with polar lipids (e.g., phospholipids and glycolipids) resulting in “Phenolipids” which have a remarkably better solubility in the oily phase than the pure phenolic compounds (Mohamed F. Ramadan (2012) Antioxidant characteristics of phenolipids (quercetin-enriched lecithin) in lipid matrices. Industrial Crops and Products, 36: 363-369—incorporated by reference in its entirety). The results are of importance for improving the biological activity, functionality and health impact of both polar lipids and phenolic compounds which could be applied in different food and pharmaceutical applications (Mohamed F. Ramadan and Mohsen M. S. Asker (2009) Antimicrobical and antivirial impact of novel quercetin-enriched lecithin. Journal of Food Biochemistry, 33: 557-571; Mohamed F. Ramadan (2008) Quercetin increases antioxidant activity of soy lecithin in a triolein model system. LWT-Food Science and Technology 41: 581-587—incorporated by reference in their entirety).

Complexes of polar lipids with single phenolic compounds and plant extracts rich in phenolics may provide a composition having bioactive properties suitable for different applications. This type of formulation may be useful, but a production process and the structure of the composition and their influence on the stability of food have to be answered before such compositions can be used.

### BRIEF SUMMARY OF THE DISCLOSURE

The foregoing paragraphs have been provided by way of general introduction, and are not intended to limit the scope of the following claims. The described embodiments, together with further advantages, will be best understood by reference to the following detailed description taken in conjunction with the accompanying drawings.

One embodiment of the disclosure relates to a composition called Phenolipids, comprising a phenolics complexed to a glycolipid, where the phenolics is a plant extract rich in phenolic compounds or one or more phenolic compound.

In another embodiment the Phenolipids is a quercetin-containing plant extract and the glycolipid is a glyceroglycolipid

In another embodiment the glycolipid is lecithin.

In another embodiment the Phenolipids comprises quercetin which is covalently bonded to lecithin.

In another embodiment the disclosure relates to a method for making a complex (Phenolipids) of a phenolics and a glycolipid, comprising combining the plant extract and the glycolipid in a solvent medium.

In another embodiment the solvent medium used in the method is selected from the group consisting of water and ethyl acetate.

### BRIEF DESCRIPTION OF THE DRAWINGS

FIG. 1 is a schematic diagram showing the difference in structure between liposomes and Phenolipids.

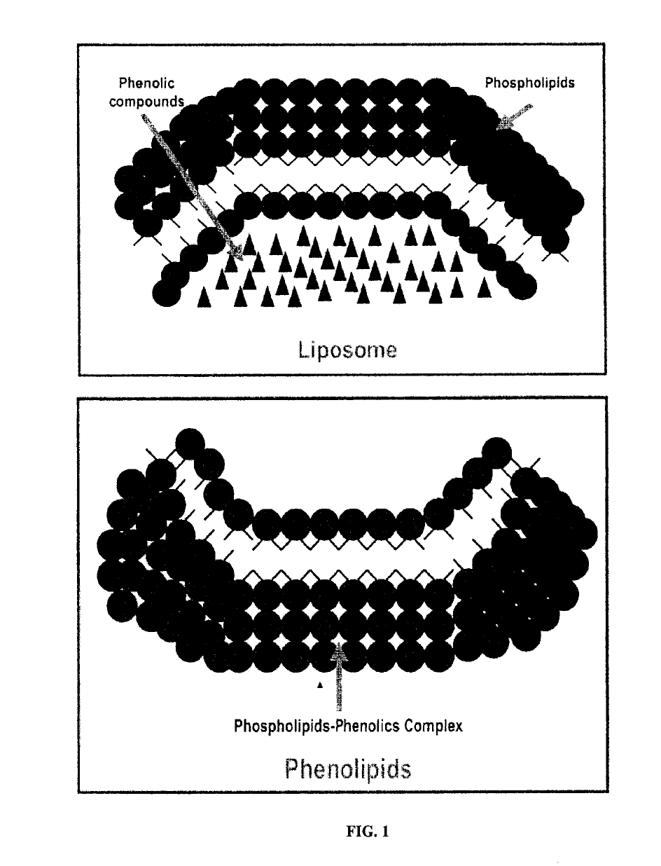

FIG. 2 is a schematic diagram showing a quercetin-phosphatidylcholine complex.

### DETAILED DESCRIPTION OF THE EMBODIMENTS

The present disclosure relates to a composition that may be used in different functional foods and pharmaceuticals. The composition of Phenolipids comprises a phenolics-containing plant extract complexed to a glycolipid. The plant extract is rich in bioactive phenolic compounds.

Phenolipid, as used herein, differ from liposomes. Liposomes are formed by mixing water-soluble substances with phospholipids, where no chemical bonds are formed. Phenolipids, unlike liposomes, result from the reaction of phospholipids with selected phenolic compounds. Phenolipids are lipophilic and freely soluble in selected solvents and in fats.

Phospholipids are polar lipids. The phospholipids of the present disclosure include phosphatidylcholine (lecithin), phosphatidic acid (phosphatidate), phosphatidylethanolamine (cephalin), phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and ceramide phosphoryllipid.

A source of polar lipids includes phospholipids from different plants and animal sources. Examples of the sources include, but are not limited to, soya, rapeseed, sunflower oil, chicken eggs, bovine milk, fish eggs, and corn oil.

A source of phenolic compounds which are not individual commercially phenolic compounds, include medicinal plant extracts. The phenolics of the present disclosure comprise a plant extract rich in phenolic compounds from at least one medicinal plant selected from the group consisting of rosemary, clove, cardamom, coriander, thyme, wintergreen, and sesame seeds.

The phenolics also contain one or more phenolic compounds, for example, are at least one selected from the group consisting of quercetin, rutin, cannabinoids, capsaicin, cresol, estradiol, eugenol, gallic acid, guaiacol, methyl salicylate, rasberry ketone, salicylic acid, serotonin, thymol, tyrosine, and sesamol.

In one embodiment, rosemary is extracted to obtain an ethyl acetate-soluble fraction, which is reacted with glycolipid to obtain a phenolipid-containing plant extract. The ethyl acetate-soluble rosemary extract can contain compounds such as Cyanidin 3,5-O-diglucoside, Luteolinidin and 6″-O-Acetyldaidzin. Rosemary may also be extracted to obtain a water-soluble fraction, which is then reacted with glycolipid to obtain a phenolipid-containing plant extract. The water-soluble rosemary extract can contain compounds such as 1,3-Dicaffeoylquinic acid, 1-Caffeoylquinic acid and 5-5′-Dehydrodiferulic acid .

In another embodiment, clove is extracted to obtain an ethyl acetate-soluble fraction, which is reacted with glycolipid to obtain a phenolipid-containing plant extract. The ethyl acetate -soluble clove extract can contain compounds such as Deoxyanthocyanins, Polymeric anthocyanins and Catechins. Clove may also be extracted to obtain a water-soluble fraction, which is then reacted with glycolipid to obtain a phenolipid-containing plant extract. The water-soluble clove extract can contain compounds such as Hydroxyphenylpropanoic acid and Hydroxycinnamic acids.

In another embodiment, cardamom is extracted to obtain an ethyl acetate -soluble fraction, which is reacted with glycolipid to obtain a phenolipid-containing plant extract. The oil-soluble cardamom extract can contain compounds such as Isoflavones, Biflavonoids and Dihydroflavonols. Cardamom may also be extracted to obtain a water-soluble fraction, which is then reacted with glycolipid to obtain a phenolipid-containing plant extract. The water-soluble cardamom extract can contain compounds such as Hydroxybenzoic acids, Hydroxycinnamic acids and Hydroxyphenylacetic acids.

In another embodiment, coriander is extracted to obtain an ethyl acetate -soluble fraction, which is reacted with glycolipid to obtain a phenolipid-containing plant extract. The ethyl-acetate-soluble coriander extract can contain compounds such as Cyanidin, Cyanidin 3,5-O-diglucoside, Butein, quercetin and rutin. Coriander may also be extracted to obtain a water-soluble fraction, which is then reacted with glycolipid to obtain a phenolipid-containing plant extract. The water-soluble coriander extract can contain compounds such as 1,3-Dicaffeoylquinic acid , caffeic acid and chlorogenic acid.

In another embodiment, thyme is extracted to obtain an ethyl acetate-soluble fraction, which is reacted with glycolipid to obtain a phenolipid-containing plant extract. The oil-soluble thyme extract can contain compounds such as quercetin and rutin. Thyme may also be extracted to obtain a water-soluble fraction, which is then reacted with glycolipid to obtain a phenolipid-containing plant extract. The water-soluble thyme extract can contain compounds such as Caffeic acid, Luteolingluc, Rosmarinic acid, and Apigenin.

In another embodiment, sesame seeds are extracted to obtain an ethyl acetate-soluble fraction, which is reacted with glycolipid to obtain a phenolipid-containing plant extract. The oil-soluble sesame seeds extract can contain compounds such as Bis-8,8′-Catechinylmethane, Dihydrokaempferol, Dihydromyricetin, and Dihydroquercetin. Sesame seeds may also be extracted to obtain a water-soluble fraction, which is then reacted with glycolipid to obtain a phenolipid-containing plant extract. The water-soluble sesame seeds extract can contain compounds such as 3,4-Dihydroxyphenylacetic acid, Homovanillic acid, and 1-Caffeoylquinic acid.

It is not necessary to purify the plant extract before reaction with the glycolipid. The plant extract can be reacted in native form. The composition of the present invention is advantageous because costly and time-consuming purification processes are not necessary. Thus, the phenolipid-comprising plant extract contains all components that are present in the plant extract. For example, the plant extract contains phenolic acids, flavonoids, quercetin and rutin.

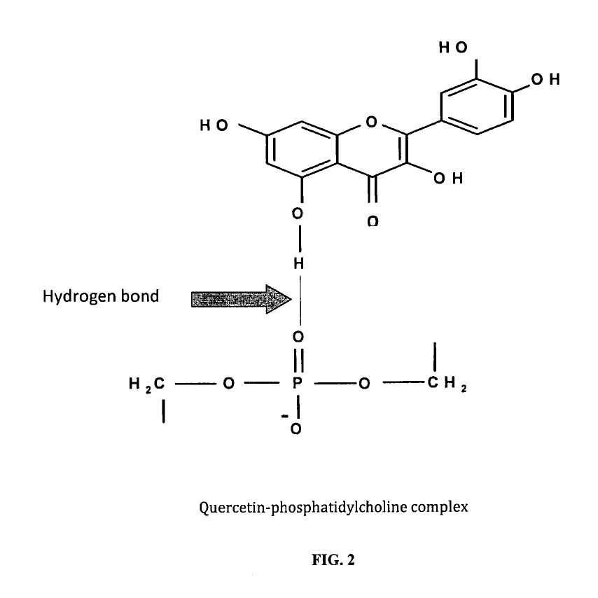

The interaction of polar lipids with phenolic compounds was studied by NMR and quantum chemistry, and revealed the ability of the phenolic compound and the polar lipids to form chain structures linked by hydrogen bonds. FIG. 2 demonstrates such a structure, where phosphatidylcholine and quercetin form a quercetin-phosphatidylcholine complex.

The phenolipid-containing plant extract of the present disclosure are prepared using safe reaction conditions and solvents under low temperature setting. A typical process for the preparation of phenolipids in accordance with the disclosure is set out in the following: reacting a phospholipid with a plant extract comprising one or more phenolic compounds in a reaction mixture comprising a solvent.

The solvent is selected such that the resultant phenolipid-containing mixture is soluble in the solvent used in the present method. The phenolipids are soluble in solvents selected from the group consisting of water and ethyl acetate.

A reaction temperature is from 30 to 50° C., preferably from 40 to 50° C., especially preferably from 45 to 50° C. If the temperature is lower than 30° C., then it is hard to dissolve polar lipids and phenolic compounds together. If the temperature is higher than 50° C., then some of polar lipids constituents might be oxidized.

A concentration of the phospholipid in the reaction mixture is from 0.5 to 1 M, especially preferably from 0.7 to 1 M. A concentration of the phenolic compound in the reaction mixture is from 1 to 3 M, especially preferably from 2 to 3 M.

A weight/weight (w/w) ratio of the phenolic compound to the phospholipid is from 1:99 to 3:97, especially preferably from 2:98 to 3:97. A weight/volume ratio of the phospholipid and the phenolic compound to the solvent is from 1:5 to 1:15, preferably 1:7 to 1:13, especially preferably from 1:9 to 1:11.

A pH of the reaction solution is from 5 to 9, preferably from 6 to 8, especially preferably from 6.5 to 7.5.

Reaction mixture including phenolic compounds (or plant extract rich in phenolic compounds) with phospholipids or glycolipids should be mixed thoroughly in the used solvent (ethyl acetate or water) and heated in a water bath with rotation for 30 min. Then the ethyl acetate should be evaporated under vacuum if ethyl acetate was used as solvent medium. If the water was used as solvent medium the aqueous solution containing Phenolipids should be freeze-dried to obtain Phenolipids in a powder form.

The plant extract in the present method does not need purification before reaction with the glycolipids. Preferably, the plant extract is reacted in its native form so as to avoid time-consuming and costly purification processes, and to obtain higher yield of the composition.

The phenolipids obtained by the present method have higher antioxidant and antimicrobial properties than corresponding compounds (phenolics or polar lipids). In addition, formulation of phenolipids is proved to enhance biological and functional properties of the phenolics and polar lipids resulting in innovative applications in novel foods, cosmetics and pharmaceuticals.

The antioxidant, antiradical and antimicrobial properties of the obtain Phenolipids are characterized herein.

Antioxidative activities of lecithin and mixtures of quercetin and lecithin (1:1, w/w) in the protection of triolein and sunflower oil models stored under accelerated oxidative conditions for 15 days in the dark at 60° C. were studied. Oxidative stabilities of quercetin-lecithin enriched models were better than in models containing lecithin or quercetin alone, most likely as a consequence of synergism between polar lipids and quercetin.

Antimicrobial and antiviral activities of quercetin-enriched lecithin formulations were found to be better than individual lecithin or quercetin, most likely as a consequence of synergism between polar lipids and quercetin. Quercetin-enriched lecithin formulations exhibited strong antimicrobial action in comparison with native lecithin and quercetin. Quercetin-enriched lecithin formulation (1:99, w/w) showed also high antibacterial activity against gram-positive bacteria with minimum inhibitory concentration (MIC) between 750 and 1000 μg/mL. Quercetin-enriched lecithin formulation (3:97 w/w) had a drastic effect on the biosynthesis of protein in cells of B. subtilis, but the effect was slightly noted on the biosynthesis of DNA and RNA.

In this disclosure, preparation of phenolipids from different polar lipids and phenolics or plant extracts (with noval biological and functional properties) is anticipated to play a vital role in efficient herbal drug delivery of a broad spectrum of protective phytochemicals. After selection of potential bioactive phytochemicals from medicinal or aromatic plants, phenolipids can be used and developed not only for specific application in food preparation but also for various therapeutic uses like cardiovascular, anti-inflammatory and anticancer activities. Moreover, phenolipids are anticipated to show their potential in cosmetics as anti-skin ageing agents and for the use of other nonpathogenic skin conditions.

Particularly, phenolipids, preferably phenolipids from lecithin and quercitin, have particular bioactive functionality superior to conventional polar lipids or phenolic compounds due the the diverse properties of polar lipids or phenolics as well as the synergism of polar lipids and phenolics.

The foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present disclosure is intended to be illustrative, but not limiting of the scope of the disclosure, as well as other claims. The disclosure, including any readily discernible variants of the teachings herein, define, in part, the scope of the foregoing claim terminology such that no inventive subject matter is dedicated to the public.

1: A phenolipids composition obtained by reacting plant extract rich in phenolics with a glycolipid, where one or more phenolic compound is complexed to the glycolipid.

2: The composition of claim 1, wherein the phenolipid-comprising plant extract and the glycolipid is a glyceroglycolipid.

3: The composition of claim 1, wherein the glycolipid is lecithin.

4: The composition of claim 1, wherein the plant extract is rich in quercetin.

5: The composition of claim 1, wherein the bioactive plant extract is from at least one medicinal plant selected from the group consisting of rosemary, clove, cardamom, coriander, thyme, wintergreen, and sesame seeds.

6: The composition of claim 1, wherein the plant extract comprises at least one phenolic compound selected from the group consisting of quercetin, rutin, cannabinoids, capsaicin, cresol, estradiol, eugenol, gallic acid, guaiacol, methyl salicylate, rasberry ketone, salicylic acid, serotonin, thymol, tyrosine, and sesamol.

7: A method for making a complex of a phenolics and a glycolipid, comprising combining a plant extract and a glycolipid in a solvent medium, wherein the plant extract comprises a phenolics.

8: The method of claim 7, wherein the solvent medium is selected from the group consisting of water and ethyl acetate.
